(19) United States
(12) Patent Application Publication
(10) Pub. No.: US 2019/0008436 A1
(43) Pub. Date: Jan. 10, 2019

(54) METHOD AND SYSTEM FOR MONITORING AND IMPROVING ATTENTION

(71) Applicant: Atentiv LLC, Beverly, MA (US)

(72) Inventors: Andrew D. KRYSTAL, Durham, NC (US); John R. SHAMBROOM, Framingham, MA (US); Stephan E. FABREGAS, Cambridge, MA (US); Brian TRACEY, Arlington, MA (US)

(73) Assignees: Atentiv LLC, Beverly, MA (US); Atentiv LLC, Beverly, MA (US)

(21) Appl. No.: 15/748,740

(22) PCT Filed: Jul. 29, 2016
(86) PCT No.: PCT/US2016/044828

§ 371 Date: Jan. 30, 2018

Related U.S. Application Data
(60) Provisional application No. 62/199,749, filed on Jul. 31, 2015.

(51) Int. Cl. A61B 5/16; A61B 5/0484; A61B 5/04; A61B 5/00

(52) U.S. Cl. CPC A61B 5/168; A61B 5/0484; A61B 5/726; A61B 5/6803; A61B 5/7257; A61B 5/04012

(57) ABSTRACT

The invention features methods and systems useful for monitoring attention. The methods and systems can be used as part of an EEG brain-to-computer interface that measures the attention level of a subject and trains the subject to improve attention.

### FIELD OF THE INVENTION

The present invention features a method and system for monitoring and training attention in subjects.

### BACKGROUND OF THE INVENTION

Attention Deficit/Hyperactivity Disorder (ADHD) is one of the most common childhood disorders, with the US CDC estimating that 11% of children between the ages of 3-17 struggle with the disorder. The underlying mechanisms and associated cognitive dysfunctions remain unclear, with several competing theories that all point to the complexity of this disorder. Children who suffer from ADHD experience problems such as lower levels of academic achievement, higher dropout rates, higher likelihood of drug abuse, diminished social relationships, and a higher rate of mental illness than non-clinical children of the same age. To date, the most efficacious and best studied treatment for ADHD remains stimulant medication. While medications have been reliably shown to improve behavior at home and in the classroom, these improvements seen after taking medication are not long-lasting. Benefits also appear to be lost after termination of use and come with many side effects, including headaches, nausea, suppressed appetite, reduction in physical growth, and cardiovascular effects. These stimulant medications are also potential drugs of abuse.

Direct monitoring of brain signals offers the ability to more specifically characterize the attention state of a user by looking at well-defined brain functions, but only if the brain signals can be processed to produce a statistically meaningful measure of attention and inattention.

There is a need for methods and systems capable of monitoring the attention state of a subject in real time, particularly in subjects suffering from disorders characterized by inattention, such as attention deficit and hyperactivity disorder (ADHD), depression, anxiety disorders, schizophrenia, or autism, and for use in attention training systems (e.g., feedforward learning).

### SUMMARY OF THE INVENTION

The invention features a method for classifying an EEG brain signal including: (i) placing, in proximity to a subject, a device connected to a computer, wherein the device can be activated by the subject; presenting to the subject instructions with respect to activating the device in response a stimulus, wherein the subject is instructed to activate the device when a specified stimulus is presented to the subject; and presenting to the subject the stimulus while recording instances of device activation by the subject; (ii) recording one or more of EEG brain signals of the subject while performing at least a portion of step (i); (iii) storing the instances of device activation by the subject from step (i) and the one or more EEG brain signals from step (ii) in a computer; (iv) determining a response time parameter of device activation and calculating response time values for each of the one or more EEG brain signals; and (v) on the basis of the response time values from step (iv), classifying the one or more EEG brain signals to produce labeled brain signals characteristic of the subject having an attentive state or an inattentive state. The method can further include classifying the one or more EEG brain signals to produce labeled brain signals characteristic of the subject having (a) an attentive state, (b) a first inattentive state; or (c) a second inattentive state characterized by a subject's level of drowsiness. In certain embodiments, the method further includes identifying the one or more EEG brain signals with increasing relative power in the delta or theta bands coincident with longer reaction times, and labelling the EEG brain signals as belonging to the second inattentive state. The method can further include calculating the subject's level of drowsiness. In particular embodiments, the method includes determining whether the subject's level of drowsiness exceeds a predetermined threshold and, if so, alerting the subject (e.g., with an alarm or image to encourage vigilance in the subject). In one embodiment of the above methods, the response time values for each of the one or more EEG brain signals are composite values calculated from the response time parameter and the EEG brain signals. In certain embodiments, step (v) includes classifying the one or more EEG brain signals by cluster analysis of the composite values. In other embodiments, step (v) includes classifying the one or more EEG brain signals by cluster analysis of the EEG brain signals and coincident response time values. In still other embodiments, the response time parameter or the response time value is age-adjusted, adjusted for gender, or adjusted for a psychiatric condition (e.g., ADHD versus normal, or subjects suffering from depression, anxiety disorders, schizophrenia, or autism). In some embodiments, the response time value is adjusted for the measured severity of a psychiatric condition in the subject (e.g., the severity of ADHD, depression, anxiety disorders, schizophrenia, or autism). In particular embodiments, the subject has ADHD and the response time value is adjusted for the measured severity of ADHD in the subject (e.g., a composite including the subject's ADHD-RS score). The response time value is coincident with EEG brain signals measured 1 to 4 seconds (e.g., 1, 1.5±0.5, 2.0±0.5, 2.0±1, or 3.0±1 seconds) immediately prior to presenting to the subject the stimulus, or immediately prior to the subject's response to the stimulus. The method can further include generating a representation of a subjects attention level including: (a) providing a generalized subject-independent model derived from electroencephalographic (EEG) brain signals from a pool of subjects, the subject-independent model including labeled brain signals; (b) providing subject-specific EEG brain signals obtained from the subject; (c) on the basis of the subject-independent model and the subject-specific brain signals, calculating a score representing the probability that the subject is attentive or inattentive; and (d) presenting the score to the subject. In particular embodiments, step (c) includes comparing the subject-specific EEG brain signals to the labeled EEG brain signals from a pool of subjects, and on the basis of the comparison determining the probability that the subject is attentive or inattentive.

In a related aspect, the invention features a method for generating a representation of a subjects attention level including: (i) providing a subject-independent model derived from electroencephalographic (EEG) brain signals from a pool of subjects, the subject-independent model including labeled brain signals associated with (a) an attentive state, (b) a first inattentive state; or (c) a second inattentive state characterized by a subject's level of drowsiness; (ii) providing subject-specific EEG brain signals obtained from the subject; (iii) on the basis of the subject-independent model and the subject-specific brain signals, calculating a score representing the probability that the subject is attentive or inattentive; and (iv) presenting the score to the subject. In particular embodiments, step (iii) includes comparing the subject-specific EEG brain signals to the labeled EEG brain signals from a pool of subjects, and on the basis of the comparison determining the probability that the subject is attentive or inattentive. The method can further include: (x1) inputting the score into a video game; (x2) presenting a video game having at least one output to the subject; (x3) presenting to the subject at least one signal corresponding to the score; and (x4) altering the difficulty or progress of the game if the score exceeds a predetermined threshold or falls outside a predetermined range.

The EEG brain signals can be processed to produce one or more EEG parameters using a method selected from Fourier transform analysis, wavelet analysis, absolute power analysis, relative power analysis, phase analysis, coherence analysis, amplitude symmetry analysis, and/or inverse EEG analysis (e.g., localization of electrical activity in the brain), or any other methods known in the art. In one embodiment of any of the above methods, the EEG brain signals can be selected from the relative power of one or more frequency bands. In another embodiment, the EEG brain signals are selected from the absolute power of one or more frequency bands. In a related aspect, the invention features a system for generating a representation of attention level in a subject including: (i) an EEG headset for collecting EEG data from the subject; and (ii) a processor equipped with an algorithm for calculating a score representing the probability that the subject is attentive or inattentive according to the methods of the invention.

As used herein, the term “response time value” refers to a response time, or a value calculated using the response time, measured when a subject is instructed to activate a device when a specified stimulus is presented to the subject while recording one or more of EEG brain signals of the subject. The response time value can be, e.g., a composite value calculated from the response time and the coincident EEG brain signals collected at the time the response time is measured. Alternatively, the response time value can be calculated from the measure response time without including any coincident EEG brain signals.

As used herein, the term “level of drowsiness” refers to the frequency or degree to which a subject is found to be in a drowsy inattentive state characterized, e.g., by increased relative power in the delta and theta EEG brain signals (e.g., relative to the power of the alpha and beta EEG signals) of the subject and/or slow response times as measured when a subject is instructed to activate a device when a specified stimulus is presented to the subject while recording one or more of EEG brain signals of the subject.

Other features and advantages of the invention will be apparent from the following Detailed Description, the drawings, and the claims.

### BRIEF DESCRIPTION OF THE DRAWINGS

FIG. 1 is an image depicting a representation of a three cluster model. In the preferred embodiment the three clusters correspond to: (i) an attentive cluster, (ii) a first inattentive cluster, and (iii) a second inattentive cluster. A similar model was generated using EEG, reaction time, and age data as described in Example 2.

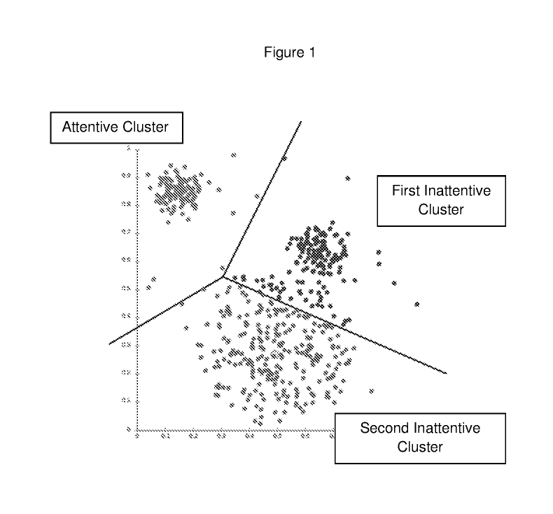

FIG. 2 is a flow chart depicting a system including a server and a local device for generating and using a real-time attention measure in a specific subject (e.g., in the performance of a game) using the methods of the invention.

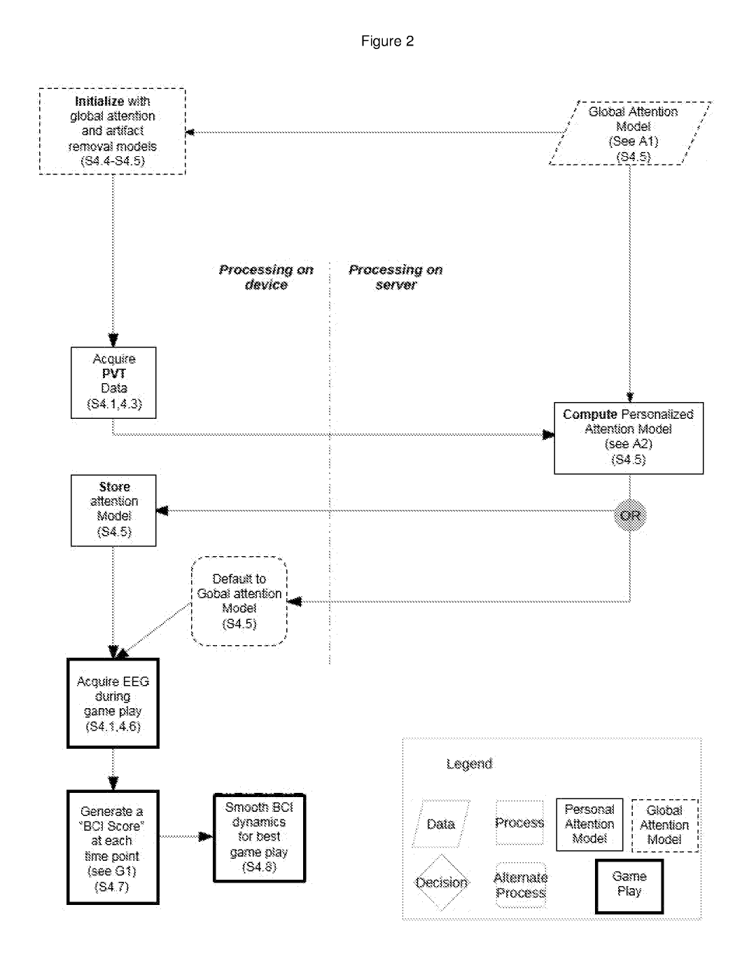

FIG. 3A is a flow chart depicting a process for creating a subject independent model from a pool of data from multiple subjects (the “training set”). FIG. 3B is a flow chart depicting a process for creating a subject-specific model of attention using the methods of the invention.

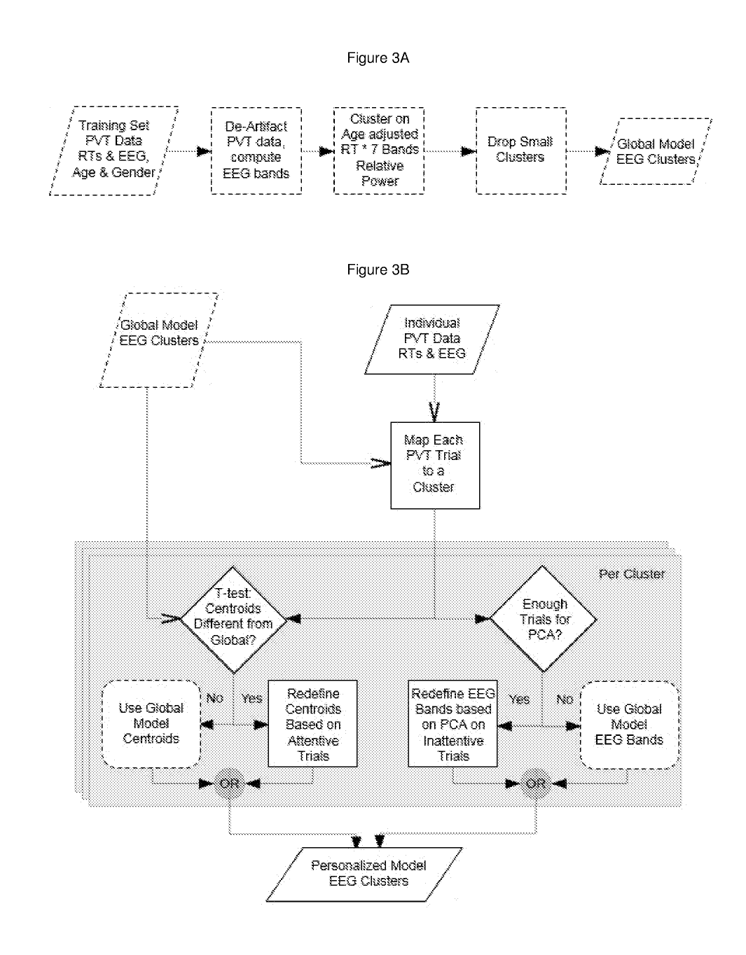

FIG. 4 is a flow chart depicting a process for creating a subject-specific attention score during gaming or other activity.

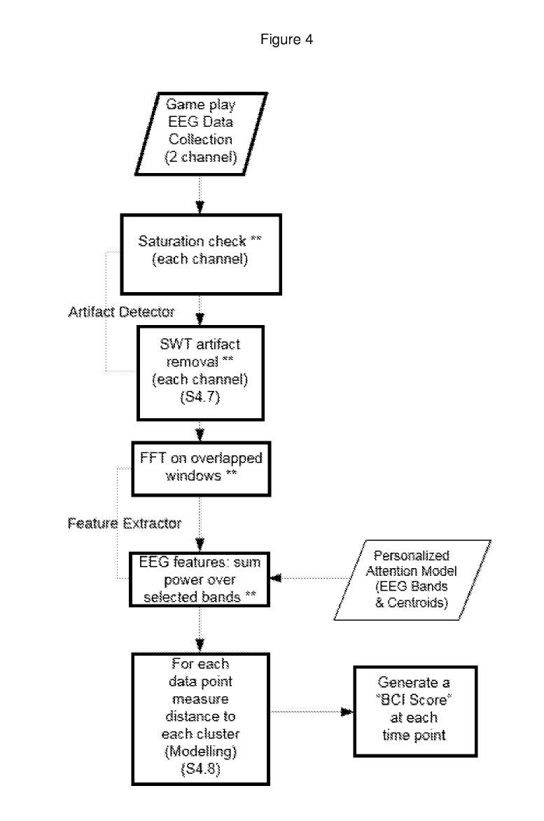

FIG. 5 is a flow chart depicting an alternative process for creating a subject independent model from a pool of data from multiple subjects (the “training set”). The drowsiness measure can be computed as described in Example 3. The Global model can include a three cluster model including (i) inattentive state characterized by a subject's level of drowsiness identified by the drowsiness measure, (ii) an attentive state, and (iii) a non-drowsy inattentive state (e.g., daydreaming inattentive). The attentive and non-drowsy inattentive EEG states can be labeled on the basis of the EEG brain signal and the coincident reaction time, or a composite thereof.

### DETAILED DESCRIPTION

The present invention features a system and apparatus for monitoring real-time attention in a subject. The methods include a calibration procedure to identify periods of time when subjects are attentive. For example, a Psychomotor Vigilance Task (PVT) can be used for the calibration procedure in conjunction with EEG data collection trials. The reaction time during each PVT trial is used as an indicator attentional state during the trial (i.e., where short reaction times suggest that the subject was attentive during the trial and slow reaction times suggest that the subject was inattentive during the trial).

The classification of EEG features based solely upon PVT reaction times would lead to errors and inconsistency (e.g., where subject are randomly responding and not paying attention, or their reaction to a prior stimulus may be so delayed that if falls in the rapid response range of the subsequent stimulus). To address this issue, the present methods and systems identify a subject as being in an attentive state when both performance (i.e., reaction time) and EEG features simultaneously indicate a state of high attention level. Thus, the present methods include classifying EEG features using a reaction time and EEG signal, or a composite thereof.

It is known that PVT reaction time is affected by age, especially in young children (Venker, et al. Sleep & Breathing, 11(4), 217-24). RT performance improves (speeds up) throughout childhood before leveling off in late adolescents. This relationship may be approximated linearly using, e.g., equation (a):

[RTadj=RT−(AgeNorm−Age)*k], (a)

where RTadj is the adjusted RT, and AgeNorm is the normative age for which no adjustment is made, and k is the adjustment factor in milliseconds per year of age. Alternatively the relationship may be approximated asymptotically using, e.g., equation (b):

$$[ RT adj = RT * ( 1 - \frac{m}{Age} ) ] , \qquad ( b )$$

where m is the adjustment factor in years. In another alternative, the adjustment may be made by means of a lookup table containing normative data over the range of ages.

As described in the Examples, we identified three groupings in our classification of EEG features: (i) those EEG features associated with inattention and characterized by long reaction times (compared to the attentive group) and EEG activity associated with drowsiness (the drowsiness group); (ii) those EEG features associated with inattention and characterized by long reaction times (compared to the attentive group) and EEG activity associated with non-drowsy inattention (the daydreaming inattention group); and (iii) those EEG features associated with attention and characterized by shorter reaction times (compared to the inattentive groups) and EEG activity associated with attention. We then use the EEG features which best discriminate these three groupings to produce a model for identifying states of attention and inattention in real-time.

EEG Data Collection

The invention features methods and systems that utilize EEG data. The EEG data can be collected, for example, using an electrode system in the form of a headset. Headsets suitable for use in the invention include those described, for example, in U.S. Ser. No. 14/179,416, incorporated herein by reference. The International 10-20 System provides for standardized electrode locations, and recently higher density systems have been developed (sometimes called the 10-10 System). The headsets of the invention can be designed to (i) intuitively and conveniently place electrical sensors at positions AF3 and AF4 (as well as a ground electrode, which optionally is placed at the mastoid) of the 10-10 system on the forehead of a child (i.e., without significant training in how to wear the headset), (ii) account for the variability in head size among children of different ages, and (iii) be comfortable to wear. For example, particular embodiments of the headsets of the invention are sized and configured to accommodate a range of head sizes from the 5th percentile of 8 year old girls to the 95^{th} percentile of 18 year old boys. While the headset of the invention is designed for kids ages 8-18, it will also fit most adults as well, since the head size of an 18 year old boy is close to adult sized head.

The headsets contain electrical sensors that measure EEG signals that are processed by an external computer. The electrical sensors can include one or more electrodes for measuring EEG signals of a user. The electrodes can be dry electrodes or wet electrodes (i.e., a dry electrode can obtain a signal without a conductive and typically wet material between the electrode and the user's skin, and a wet material does require such a conductive material). The electrical sensor can include a dry electrode, such as a dry fabric electrode. Fabric electrodes suitable for use in the methods and systems of the invention include those described in U.S. Patent Pub. No. 20090112077, incorporated herein by reference. The electrical sensors can contain padding to aid in the comfort of the user and also aid in adjustability and improving skin contact.

The collected EEG data is transferred to a computer for processing as described herein.

EEG Processing

The methods and systems of the invention utilize multichannel EEG acquisition to collect data from various frequency bands of a subject's brain activity to distinguish between attention states. Relatively greater beta (approximately 16-32 Hz) activity has been observed in vigilant states, whereas alpha (approximately 8-16 Hz) activity predominates in alert but less mentally busy states, and theta (approximately 4-8 Hz) activity rises as attention lapses (Streitberg et al., Neuropsychobiology Vol 17, 105-117, 1987). Optionally, the methods of the invention can be performed without decomposing the EEG data into frequency bands. For example, EEG data could be transformed from frequency to time domain data, where the EEG features used in the methods of the invention have a particular width. Alternatively, phase-space based analytic procedures could be utilized to identify EEG features characteristic of attention or inattention.

In addition to distinct frequency bands, the method can include quantification of EEG signals at distinct recording sites at the brain. In one embodiment, the voltage difference is measured between the AF3 and AF4 electrodes, which sense electrical activity in the dorsal anterior cingulate cortex. In studies utilizing functional magnetic resonance imaging (fMRI) has been observed that the dorsal anterior cingulate cortex becomes active when attention lapses (Uddin et al., Journal of Neuroscience Methods, 169:249 (2008)). Thus, monitoring the brain signals obtained from that region should be informative when children with ADHD use a headset including sensors at AF3 and AF4. The temporal lobes have been implicated in some forms of ADHD, therefore some embodiments include an electrode on one or both of the mastoid processes (Rubia et al., Biological Psychiatry, 62:999 (2007)).

The EEG channels are denoised to remove non-EEG artifacts such as eye blinks and movements, muscle activities, etc. This denoising step is necessary to avoid introduction of substantial artifacts into the subsequently derived EEG features. Denoising can be performed according to known wavelet transform techniques (see, e.g., Zikov et al., Engineering in Medicine and Biology, 2002. 24th Annual Conference and the Annual Fall Meeting of the Biomedical Engineering Society EMBS/BMES Conference, 2002. Proceedings of the Second Joint. Vol. 1. IEEE, 2002). In the preferred embodiment the denoised EEG channels are normalized to produce measures of power relative to the total power over all bands. Details are provided in the Examples.

Global Model of Attention and Inattention

A global model is generated using the EEG components resulting from pre-processing. The global model is a subject-independent model which is based on data from a large number of individuals. Calibration for each subject is carried out to allow fine tuning of this model in order to improve the ability to discriminate that subject's attentive and inattentive states.

A global model can be derived through the integration of pre-processed components with additional relevant parameters. In one embodiment, the global model can include factors such as age, reaction time (RT), in addition to EEG features, the latter two obtained from a psychomotor vigilance task (PVT, described below) (Dinges & Powell, Behavioral Research Methods, Instrumentation, and Computers, 17:652 (1985)). These additional parameters are operative to provide model development that better discriminates attentive and inattentive states. Optionally, pre-processed components are multiplied by age to weight each EEG feature profile. In another embodiment pre-processed components are multiplied by corresponding RT to weight each EEG feature profile. In one embodiment, pre-processed EEG components are multiplied by both age and corresponding RT. This gives the subsequent analysis the freedom to explore the interaction of age, RT, and the EEG feature profile. In another embodiment RT is adjusted by age to account for age related changes in RT. In another embodiment the pre-processed components are multiplied by ADHD-RS score, giving the subsequent analysis the freedom to explore the interaction of ADHD severity with the other variables.

These RT-weighted variables can be further normalized through the use of Z-transformation, in preparation for subsequent principle component analysis, which is sensitive to relative differences in sizes of variables. Composite values can be used to describe the variance accounted for by EEG features in terms of discriminating attentive and inattentive state. Alternatively, the variance can be accounted for on the basis of the EEG features and coincident reaction time values. In one embodiment, this operation involves a principle component analysis and subsequent cluster analysis. A principle component analysis can be performed to generate a set of potential discriminating variables which are orthogonal (uncorrelated) thereby preventing problems with multi-colinearity in model development. In another embodiment, segments containing EEG indications of drowsiness (elevated delta and/or theta activity) are first labelled and separated from the dataset, and a subsequent logistic regression is performed on the remaining dataset. The regression separates instances of attentiveness from inattentiveness on a continuum. Additional details are provided in the Examples.

Subject-Specific Model and Classification of EEG Data

Although a subject's brain activity within distinct frequency bands correlates with his or her attention state as described above, there are significant differences in brain activity profiles between subjects. The relative powers in the set of frequency bands that discriminate best among states of attentiveness for one individual may not be precisely the same as for another individual. Therefore, to derive an EEG index of attention with which to assess mental engagement in a task, the development of a subject-dependent EEG-to-state mapping profile, herein referred to as the subject-dependent model, may provide a more accurate representation of the specific subject's attentiveness.

One aspect of the current invention relates to the personalization of the algorithm to individual users. In one aspect of the invention, each individual user begins by undergoing a PVT task with simultaneous EEG measurement. Pre-processing of EEG features is performed as described above, and the data from the PVT trials are mapped onto the principle component-defined space from the global model above. A cluster analysis is performed on the individual subject's data, and the centroids of these clusters are compared to those of the global model. Using a logistic regression operation, a probability of a user having the attention state associated with one of the clusters is derived as described below. In another aspect of the invention, an individual subject's brain state is monitored outside the context of a PVT. In this embodiment, the subject's EEG features are mapped to clusters derived from the RT-independent protocol described above. Additional details are provided in the Examples.

Use of the Model to Monitor Attention and Inattention in Subjects

The methods and systems of the invention permit a real-time determination of a probability of a subject having a particular attentive state. Following the cluster analysis or logistic regression measured EEG values derived from the EEG recorded during a given interval of time are entered into the subject specific model and used to compute the probability of attention. Additional details are provided in the Examples.

Applications

The methods and system of the invention can be used for monitoring the attention levels of any individual performing a task that requires attentiveness. The attention level of the subject can be detected, recorded, and analyzed to determine whether the subject is attentive. If the subject is observed to be inattentive, the subject may be prompted to pay attention. Optionally, a third party (e.g., maybe a teacher or parent), may be alerted to the attention status of the subject.

The methods and system of the invention can be used for training attention by providing a real-time measure of attention level in a subject undergoing training. For example, the methods and systems of the invention can be incorporated into a training system, such as a feedforward training system, to improve attention in a subject.

One aspect of the invention relates to the use of the output value to direct a video game, which is controlled by the subject. Preferably, the means for generating and displaying the video animation further includes means for maintaining the video animation while the measured electrical activity is simultaneously being processed. Furthermore, the processing means is capable of storing the electrical activity measurement and comparing the measurement with a global model.

In certain embodiments, elements of the video game are controlled by the subject's attention state. Preferably, this controlling is continuously performed by the method of the invention as the attention level changes, rather than at specified attention states. The subject is thus encouraged to maintain appropriate levels of attention in order to succeed in playing the game.

Systems for Attention Training

The methods and systems of the invention can be integrated as part of larger system to for attention measurement and training. The system can include an EEG headset device for monitoring the brain function of the subject. The headset device can provide input to a training program operating on a computer equipped with a software package. The system additionally can include a server, onto which the training program software is stored, or the global model is stored. Data can be processed on the server, on the computer, and/or on the headset device. Data detected by the headset and processed through the training program are presented to the subject through an electronic interface, such as a visual display. Displays can be disposed in the field of view of the subject to provide continuous information derived from the subject's EEG data. The electronic interface can be housed in a device such as a personal desktop computer, laptop, tablet, smartphone, or gaming system.

The following examples are put forth so as to provide those of ordinary skill in the art with a complete disclosure and description of how the methods and systems claimed herein are performed, made, and evaluated, and are intended to be purely exemplary of the invention and are not intended to limit the scope of what the inventors regard as their invention.

### Example 1: Collection of EEG Annotated with Reaction Time Data

A psychomotor vigilance task (PVT), which measures a subject's reaction time to a stimulus, was administered to subjects while simultaneously recording the subjects' brain activity. This process was used to obtain information about whether a given set of EEG features at any instance are associated with attentiveness or inattentiveness. Measures of attention other than PVT may also be used. The PVT is also useful for eliciting states of attentiveness or inattentiveness in a subject during the data collection. This is achieved by administering stimuli at various intervals over a long period of time, during which the subject must attempt to remain vigilant in attending to the task. Instances of lased attention tend to result in longer reaction times, and such instances may become more frequent over time.

The PVT was administered through a touch-sensitive video monitor as follows: A light stimulus appeared at random intervals of 2 to 10 seconds and the subject is directed to touch the screen as fast as possible following the stimulus. This is carried out over a 10 minutes period. Reaction time was measured and recorded for each trial. Approximately 80 to 100 reaction times were collected for each subject.

The EEG profile of a one-second segment immediately prior to a stimulus was selected for analysis in combination with the PVT reaction time. This time segment represents a relatively quiescent state that provides an indicator of the subject's brain state at the time of the stimulus without being affected by the subject's response to the stimulus. The complete response includes visualizing, remembering, intending, and acting.

EEG features were extracted from each one-second segment. The features can include the power in each of 7 frequency bands each divided by the total power across all bands, thus representing relative power in each band for a given PVT trial associated with a trial reaction time. EEG data was collected at two channels (AF3 and AF4), resulting in 14 frequency features for a given PVT reaction time.

### Example 2: EEG Classification by Cluster Analysis

The EEG and PVT reaction times data were used to classify EEG features as characteristic of states of attention and inattention observed in the subjects during the course of the testing described in Example 1.

The EEG and PVT reaction times data were pooled. Each EEG feature in the pooled data was multiplied by the reaction time for its trial and also multiplied by the age of the specific subject to create a variable for analysis (the general form of equation is shown in equation 1).

Iba_theta_mastoid_r=latency*b_theta_mastoid_r*age (1),

where Iba_theta_mastoid_r is the composite variable, latency is the reaction time, b_theta_mastoid_r is the relative EEG power in the mastoid channel frequency range of 4-8 Hz.

Each variable type was Z-transformed across the entire pool of like variables (the general form of equation is shown in equation 2).

Iba_theta_mastoid_rz=(Iba_theta_mastoid_r−E)/F (2),

where Iba_theta_mastoid_rz is the Z-transformed composite variable, and E and F are constants derived in the analysis.

Next the variables were submitted for principal components analysis (PCA). All or a subset of variables may be submitted for analysis. For example, equation 3 (below) could be used, where A thru D are constants derived from the PCA.

prin2=A*Iba_delta_frontal_rz+B*Iba_theta_mastoid_rz−C*Iba_alpha_frontal_rz−D*Iba_alpha_mastoid_rz−D*Iba_theta_frontal_rz (3),

where A, B, C, and D are constants derived in the analysis.

Principal components were chosen for clustering. Several methods may be used for choosing, including choosing the two that explain the most variance in the data (which would be PCAs 1 and 2). Instead, to create our model, we chose principal components that allowed the formation of clusters that best fit our model where there was one cluster that had long reaction times associated with increased high frequency activity in the default mode network, one cluster that was associated with significantly longer reaction times associated with an increase in delta and theta frequency EEG activity and a third cluster of trials that had significantly shorter reaction times than the other two groups and less default mode network high frequency activity than the first group and less theta and delta power than the second group.

The two principal components were submitted for cluster analysis along with the pooled data using SAS software. K-means clustering was performed. The clustering can be conducted once or iteratively to optimize the resulting model of attention. The result of the clustering analysis is a model having at least two clusters with their centroid coordinates (attention and inattention) defined. In this study, the best observed fit produced three centroid coordinates (one centroid for attention, and two centroids for inattention).

### Example 3: EEG Classification by Logistic Regression

The EEG and PVT reaction times data are used to classify EEG features as characteristic of states of attention and inattention observed in the subjects during the course of the testing described in Example 1.

The EEG and PVT reaction times data are pooled. The relative powers of the EEG bands (b_delta_mastoid_r, b_theta_mastoid_r, etc.) are examined for evidence of EEG slowing (i.e., increasing power in the delta and theta bands), coincident with longer reaction times, indicating drowsiness. A composite measure is created, and a threshold assigned. If the composite measure exceeds the threshold the trial is assigned to the inattentive drowsy group.

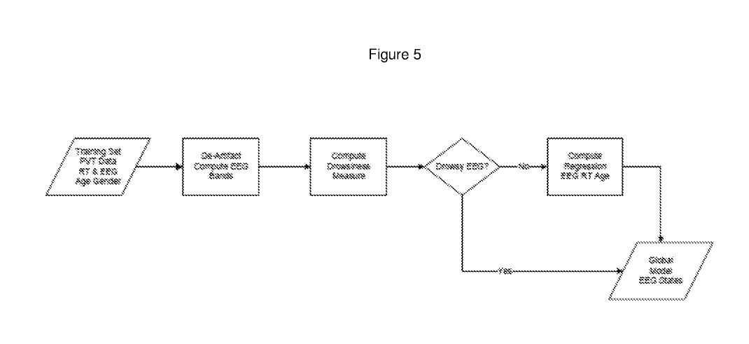

The remaining trials are subject to logistic regression to find the EEG features' correlates of reaction time (see FIG. 5). The correlation coefficients (e.g. Pearson's r) are examined, and the features with the weaker correlates are removed from further analysis. The remaining EEG features are weighted and combined to form a composite measure. The resulting measure is normalized to create an index of attentiveness.

### Example 4: A Real-Time Global Model of Attention and Inattention

The EEG and reaction time data classification from Example 2 was used to create a real-time global model of attention and inattention based solely upon EEG data collected outside of a PVT task environment.

The goal is to produce a model suitable for use in assessing EEG features as a means of indicating attention level in real-time while the subject is engaged in some way where ability to gauge attention is of utility (e.g., to assist with learning, or some other task, or no particular task). For such applications, the EEG segments would not need to be accompanied by other measures of attention, such as PVT reaction time values.

To use the aforementioned cluster analysis classification from Example 2, a method was developed using EEG data alone for assigning new data points that do not include reaction time data to the clusters.

Each feature in the pooled data set was multiplied by the age of the specific subject to create a variable for analysis (the general form of equation is shown in equation 4).

Iba_theta_mastoid_r=b_theta_mastoid_r*age (4)

Each variable type was Z-transformed across the entire pool of like variables (the general form of equation is shown in equation 5).

ba_theta_mastoid_rz=(ba_theta_mastoid_r−G)/H (5)

Next the variables were submitted for principal components analysis (PCA). All or a subset of variables may be submitted for analysis.

A logistic regression was conducted that maps the principal components not containing reaction time onto the clusters created with reaction time, along with age and EEG measures. This results in an equation that yields the probability that then EEG data collected during a segment of time indicates that the subject was in a particular attention cluster during that time segment (the general form of equation is shown in equation 6).

test_clus1=J+K*prin1+L*prin2−M*prin3+1.4302*prin4−N*prin5 (6),

where test_clus1 is the probability of membership in cluster 1, print-5 are principal components 1 through 5 resulting from the PCA analysis.

This is the form of equation used to classify new EEG data for real-time attention level monitoring.

A variety of global models can be generated according to different groupings of subjects. For example, separate global models could be derived for subjects from the age of 8-12, and 13-18 to better capture the contribution of, for example, RT, to the data variance, or for subject pooled by condition (e.g., ADHD or ADD).

Alternatively, the EEG and reaction time data classification from Example 3 could be used to create a real-time global model of attention and inattention based solely upon EEG data collected outside of a PVT task environment.

### Example 5: Evaluation of Real-Time Attention in a Subject

The model of attention from Example 4 was used to evaluate real-time attention states in subjects during EEG signal monitoring. EEG features were collected from individuals and applied to the logistic regression equation formed in the global model of Example 4 (or a subject-specific model, if desired) to calculate the proximity or distance of the weighted features from the current time-window to each of a set of pre-defined cluster centers. These distance scores are then converted into a likelihood of attentive or inattentive state based on the relative distance from the attentive cluster center and the inattentive cluster centers. This process can then be repeated over a series of discrete or overlapping time-windows in order to provide a score for attention level at any given moment in time. This process may occur in relative real-time or as a post-processing technique. Additional details are provided below.

As described above, the model of attention based upon cluster analysis was produced using one centroid (i.e., one state) characteristic of attention and two centroids (i.e., two states) characteristic of inattention, with one inattentive state being an inattentive but non-drowsy state and the other being a drowsy state (this approach can easily be extended to an arbitrary number of attentive or inattentive states). First, we calculated that the subject is in inattentive state 1 with probability p1 and in inattentive state 2 with probability p2. Then, an attentiveness index I_att was made with any function f of the form of equation 7.

I_att=f(p1,p2) (7)

In equation 7, the output of f is bounded to lie between 0 and 1, ranging from low attention to high attention (as will be obvious, the value of I_att can be scaled or transformed as desired before it is used or presented). For example, f(p1,p2)=1−max(1, p1+p2). The score was computed using a transformed and linearly weighted function of the probabilities as input to an exponential, for example, f(p1,p2)=1−exp(a0+a1*In(p1+p2)), where values of a0 and a1 are chosen to keep I_att within bounds while maximizing the discrimination power of the index across a particular dataset.

The form of the mapping procedure for children allows tailoring with respect to a child's normal or attention deficit abilities.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for classifying an EEG brain signal comprising:
(i) placing, in proximity to a subject, a device connected to a computer, wherein the device can be activated by said subject; presenting to said subject instructions with respect to activating said device in response a stimulus, wherein said subject is instructed to activate said device when a specified stimulus is presented to said subject; and presenting to said subject said stimulus while recording instances of device activation by said subject;
(ii) recording one or more of EEG brain signals of the subject while performing at least a portion of step (i);
(iii) storing the instances of device activation by said subject from step (i) and the one or more EEG brain signals from step (ii) in a computer;
(iv) determining a response time parameter of device activation and calculating response time values for each of said one or more EEG brain signals; and
(v) on the basis of the response time values from step (iv), classifying said one or more EEG brain signals to produce labeled brain signals characteristic of the subject having an attentive state or an inattentive state.

2. The method of claim 1, further comprising classifying said one or more EEG brain signals to produce labeled brain signals characteristic of the subject having (a) an attentive state, (b) a first inattentive state; or (c) a second inattentive state characterized by a subject's level of drowsiness.

3. The method of claim 2, further comprising identifying said one or more EEG brain signals with increasing relative power in the delta or theta bands coincident with longer reaction times, and labelling the EEG brain signals as belonging to the second inattentive state.

4. The method of claim 3, further comprising calculating the subject's level of drowsiness.

5. The method of claim 4, further comprising determining whether the subject's level of drowsiness exceeds a predetermined threshold and, if so, alerting the subject.

6. The method of any one of claims 1-5, wherein the response time values for each of said one or more EEG brain signals are composite values calculated from said response time parameter and said EEG brain signals.

7. The method of claim 6, wherein step (v) comprises classifying said one or more EEG brain signals by cluster analysis of said composite values.

8. The method of any one of claims 1-5, wherein step (v) comprises classifying said one or more EEG brain signals by cluster analysis of said EEG brain signals and coincident response time values.

9. The method of any one of claims 1-8, wherein said response time parameter or said response time value is age-adjusted, adjusted for gender, or adjusted for a psychiatric condition.

10. The method of claim 9, wherein said subject has ADHD and said response time value is adjusted for the measured severity of a psychiatric condition in the subject.

11. The method of any one of claims 1-10, wherein said response time value is coincident with EEG brain signals measured 1 to 4 seconds prior to presenting to said subject said stimulus.

12. The method of any one of claims 1-11, further comprising generating a representation of a subjects attention level comprising:
(a) providing a subject-independent model derived from electroencephalographic (EEG) brain signals from a pool of subjects, the subject-independent model comprising labeled brain signals;
(b) providing subject-specific EEG brain signals obtained from the subject;
(c) on the basis of the subject-independent model and the subject-specific brain signals, calculating a score representing the probability that the subject is attentive or inattentive; and
(d) presenting the score to the subject.

13. The method of claim 12, wherein step (c) comprises comparing said subject-specific EEG brain signals to the labeled EEG brain signals from a pool of subjects, and on the basis of said comparison determining the probability that the subject is attentive or inattentive.

14. A method for generating a representation of a subject's attention level comprising:
(i) providing a subject-independent model derived from electroencephalographic (EEG) brain signals from a pool of subjects, the subject-independent model comprising labeled brain signals associated with (a) an attentive state, (b) a first inattentive state; or (c) a second inattentive state characterized by a subject's level of drowsiness;
(ii) providing subject-specific EEG brain signals obtained from the subject;
(iii) on the basis of the subject-independent model and the subject-specific brain signals, calculating a score representing the probability that the subject is attentive or inattentive; and
(iv) presenting the score to the subject.

15. The method of claim 14, wherein step (iii) comprises comparing said subject-specific EEG brain signals to the labeled EEG brain signals from a pool of subjects, and on the basis of said comparison determining the probability that the subject is attentive or inattentive.

16. The method of any one of claims 12-15, further comprising:
(x1) inputting the score into a video game;
(x2) presenting a video game having at least one output to the subject;
(x3) presenting to the subject at least one signal corresponding to the score; and
(x4) altering the difficulty or progress of the game if the score exceeds a predetermined threshold or falls outside a predetermined range.

17. The method of any one of claims 1-16, wherein said EEG brain signals are processed to produce one or more EEG parameters using a method selected from Fourier transform analysis, wavelet analysis, absolute power analysis, relative power analysis, phase analysis, coherence analysis, amplitude symmetry analysis, and/or inverse EEG analysis.

18. The method of claim 17, wherein said EEG brain signals are selected from the relative power of one or more frequency bands.

19. The method of claim 17, wherein said EEG brain signals are selected from the absolute power of one or more frequency bands.

20. A system for generating a representation of attention level in a subject comprising:
(i) an EEG headset for collecting EEG data from the subject; and
(ii) a processor equipped with an algorithm for calculating a score representing the probability that the subject is attentive or inattentive according to any one of claims 12-19.
